(19) United States
(12) Patent Application Publication
(10) Pub. No.: US 2026/0151027 A1
(43) Pub. Date: Jun. 4, 2026

(54) PORTABLE OPTOMETRY DEVICE

(71) Applicant: VP Soft Co., Ltd., Daejeon (KR)

(72) Inventor: Kyung-tae YOON, Goyang-si (KR)

(21) Appl. No.: 19/348,787

(22) Filed: Oct. 3, 2025

(30) Foreign Application Priority Data
Dec. 4, 2024 (KR) 10-2024-0178705

(51) Int. Cl. A61B 3/00; A61B 3/032; A61B 3/14

(52) U.S. Cl. CPC A61B 3/005; A61B 3/0033; A61B 3/032; A61B 3/14; A61B 2560/0431

(57) ABSTRACT

Provided is a portable optometry device including: an optometry case including an internal accommodation space and a face support part configured to be seated while covering a user's eyes; a display unit arranged on one side of the internal accommodation space of the optometry case and configured to provide a screen for an eyesight test; reflection units configured to reflect a screen light output from the display unit toward the user's eye; a user input unit configured to receive a user command; and a control unit configured to store an eyesight test image including an eyesight test guidance for the user, and to control the display unit to output the eyesight test image when the user command for the eyesight test is input via the user input unit.

### BACKGROUND OF THE INVENTION

### Field of the Invention

The present invention relates to a portable optometry device, and more specifically, to a portable optometry device that is individually portable.

### Background Art

Due to modern social phenomena that people frequently use computers and smart devices, the population requiring correction of visual defects such as myopia, astigmatism, and presbyopia caused by macular degeneration and vision problems is increasing. When eyesight needs to be checked or an ocular examination is required, people typically visit an ophthalmology clinic or an optician to undergo vision and eye examinations using optometry instruments. Based on the examination results, appropriate prescriptions for corrective devices such as contact lenses or eyeglasses suitable for users are provided, or surgical and medical solutions are offered for vision correction or treatment of eye diseases.

However, individuals with limited mobility, such as persons with disabilities, the elderly, residents of remote areas, and patients who require frequent eye examinations, may find it difficult to visit an ophthalmology clinic or an optician.

Conventional optometry instruments are typically large and long, requiring a dedicated table for installation and an additional space for machine setup, resulting in limitation in mobility. Such instruments are expensive medical devices that can cost tens of millions of won.

To overcome the problems and limitations, a head-mounted, portable optometry device has been developed that can be used by anyone to perform eyesight tests by mounting a smartphone.

### PATENT LITERATURE

### Patent Documents

Patent Document 1: Korean Patent No. 10-2572093 (Optometry system)

### SUMMARY OF THE INVENTION

Accordingly, the present invention has been made to solve the above-mentioned problems occurring in the prior arts, and it is an objective of the present invention to provide a portable optometry device that is compact in size and length and is easy in portability, thereby enabling contactless (untact) eye examinations without the need to visit an ophthalmology clinic or an optician.

To accomplish the above object, according to the present invention, there is provided a portable optometry device including: an optometry case including an internal accommodation space and a face support part configured to be seated while covering a user's eyes; a display unit arranged on one side of the internal accommodation space of the optometry case and configured to provide a screen for an eyesight test; reflection units configured to reflect a screen light output from the display unit toward the user's eye; a user input unit configured to receive a user command; and a control unit configured to store an eyesight test image including an eyesight test guidance for the user, and to control the display unit to output the eyesight test image when the user command for the eyesight test is input via the user input unit. Thus, the portable optometry device can secure a viewing distance by reflecting an eyesight test image output from a display unit by a reflection unit to reduce the size and length of the optometry device compared to conventional optometry instruments, thereby enhancing user portability.

Here, the portable optometry device further includes a speaker configured to output the eyesight test guidance as voice, wherein the control unit controls the speaker to output the eyesight test guidance as voice corresponding to the portion where the eyesight test guidance is displayed in the eyesight test image. Thus, the portable optometry device can improve the convenience of the user during the eyesight test.

Moreover, the plurality of reflection units are arranged at multiple positions within the internal accommodation space of the optometry case, and the control unit controls the display unit to output a predetermined screen light intensity of the display unit corresponding to the distance of each of the plurality of reflection units. Thus, the portable optometry device includes the plurality of reflection units, thereby increasing the intensity of screen light output in proportion to the distance the screen light of the eyesight test image travels to reach the user's eyes.

Here, the portable optometry device further includes a test camera arranged within the internal accommodation space of the optometry case and configured to capture an image of the user's eye, wherein the control unit stores a plurality of disease images for various ocular diseases, compares the eye image captured by the test camera with the plurality of disease images, and identifies a disease image having a similarity greater than a predetermined threshold. Thus, the portable optometry device can capture images of the user's eye during the eyesight test, and identify ocular diseases using the captured eye image.

Furthermore, the portable optometry device further includes a lens unit which is arranged on an output path of the screen light from the display unit and includes a convex lens and a concave lens configured to enlarge and reduce the eyesight test image. So, although it may be difficult to secure a sufficient distance for the screen light of the eyesight test image to reach the user's eyes due to a small internal accommodation space, such distance can be effectively secured by using a lens unit.

Here, the portable optometry device further includes a lens moving unit configured to move the convex lens and the concave lens, wherein the convex lens and the concave lens are movable with respect to the optometry case, and the control unit controls the lens moving unit to adjust an object size in the eyesight test image for the eyesight test. Thus, the portable optometry device can enlarge and reduce object sizes in the eyesight test image by moving the lens.

In addition, the portable optometry device further includes a microphone configured to collect a user's voice, wherein the control unit stores at least one voice data corresponding to each of a plurality of objects in the eyesight test image, controls the output of the eyesight test image, compares the voice data corresponding to the output object with the user's voice collected by the microphone, and controls the device to display a next smaller object if the comparison result indicates a match, thereby allowing the user to conveniently perform an eyesight test using voice.

According to the present invention, the portable optometry device can secure a viewing distance by reflecting an eyesight test image output from a display unit by a reflection unit to reduce the size and length of the optometry device compared to conventional optometry instruments, thereby enhancing user portability.

In addition, the portable optometry device can provide an eyesight test guidance via voice output, thereby improving user convenience during the test.

Furthermore, the portable optometry device includes the plurality of reflection units, thereby increasing the intensity of screen light output in proportion to the distance the screen light of the eyesight test image travels to reach the user's eyes.

Moreover, the portable optometry device can capture images of the user's eye during the eyesight test, and identify ocular diseases using the captured eye image.

Additionally, although it may be difficult to secure a sufficient distance for the screen light of the eyesight test image to reach the user's eyes due to a small internal accommodation space, such distance can be effectively secured by using a lens unit.

In addition, the portable optometry device can show the user objects while enlarging and reducing object sizes in the eyesight test image by moving the lens, thereby allowing for an accurate eyesight test.

Furthermore, since the user reads the objects displayed in the eyesight test image, the eyesight test can be conducted smoothly and conveniently.

### BRIEF DESCRIPTION OF THE DRAWINGS

FIG. 1 is a view illustrating a user wearing a portable optometry device according to the present invention.

FIG. 2 is a view illustrating a screen light of an eyesight test image from a display unit reaching the user's eye.

FIG. 3 is a view illustrating an example of providing eyesight test guidance via voice.

FIG. 4 is a view illustrating a test camera for capturing the user's eye.

FIG. 5 is a view illustrating the operation that a lens moves.

FIG. 6 is a view illustrating an eyesight test conducted using voice.

FIG. 7 is a control block diagram of the portable optometry device.

### DETAILED DESCRIPTION OF THE PREFERRED EMBODIMENT

Hereinafter, a portable optometry device 1 according to a preferred embodiment of the present invention will be described in detail with reference to the accompanying drawings.

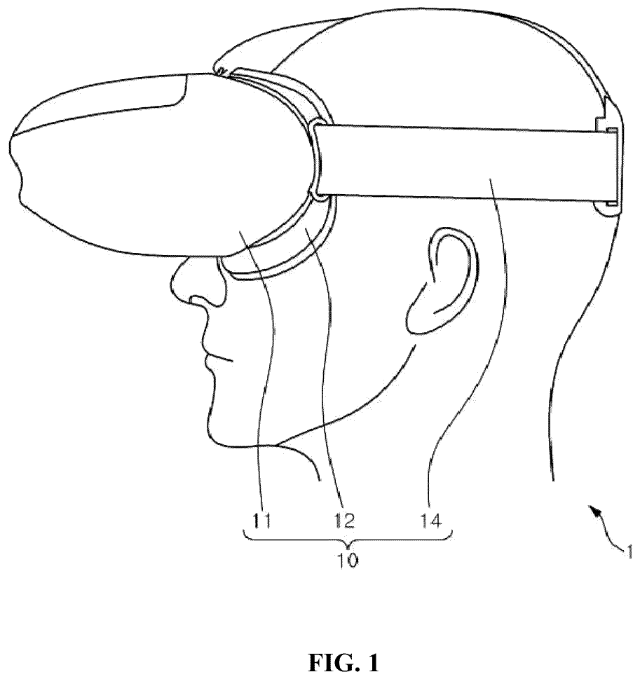

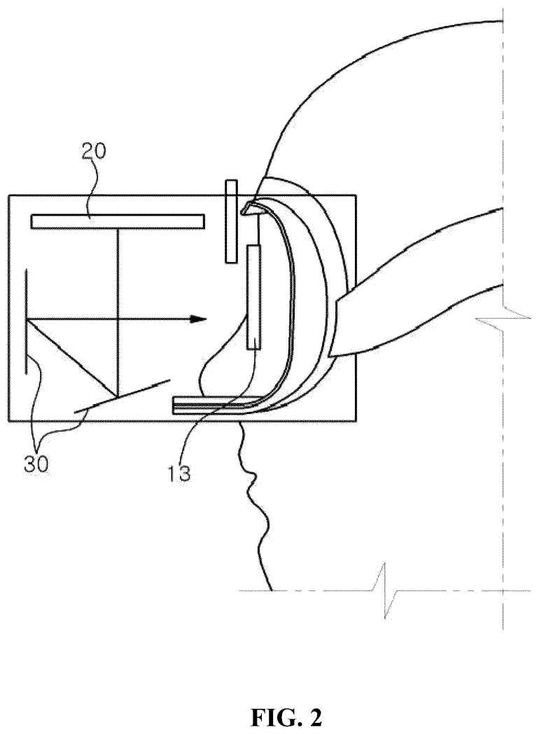

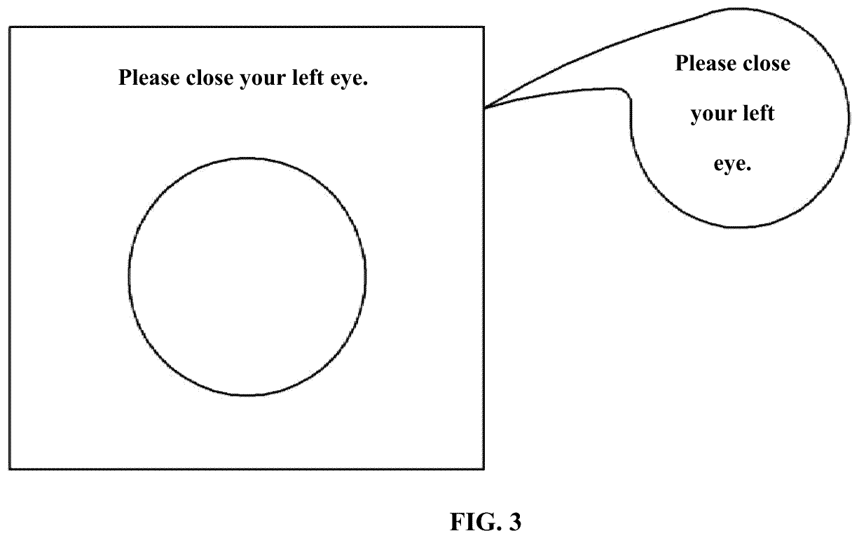

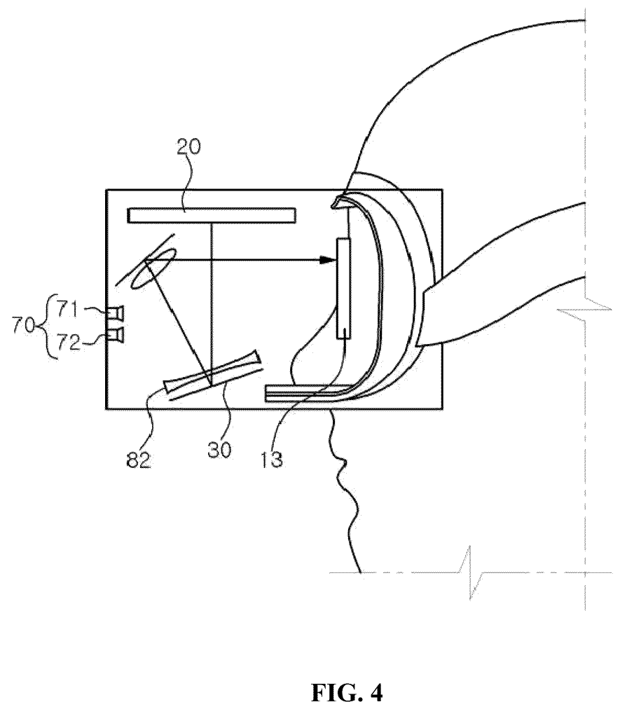

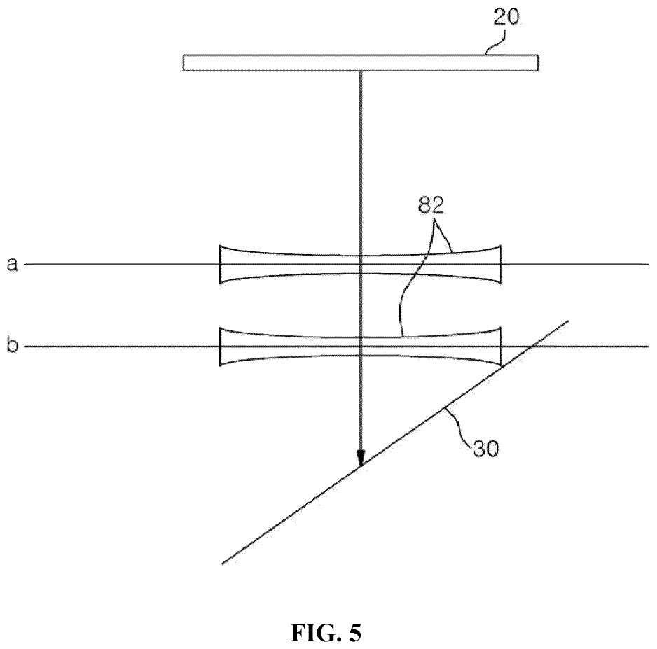

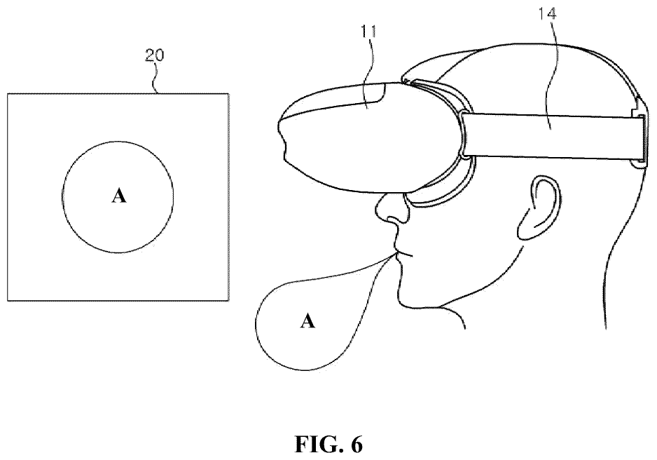

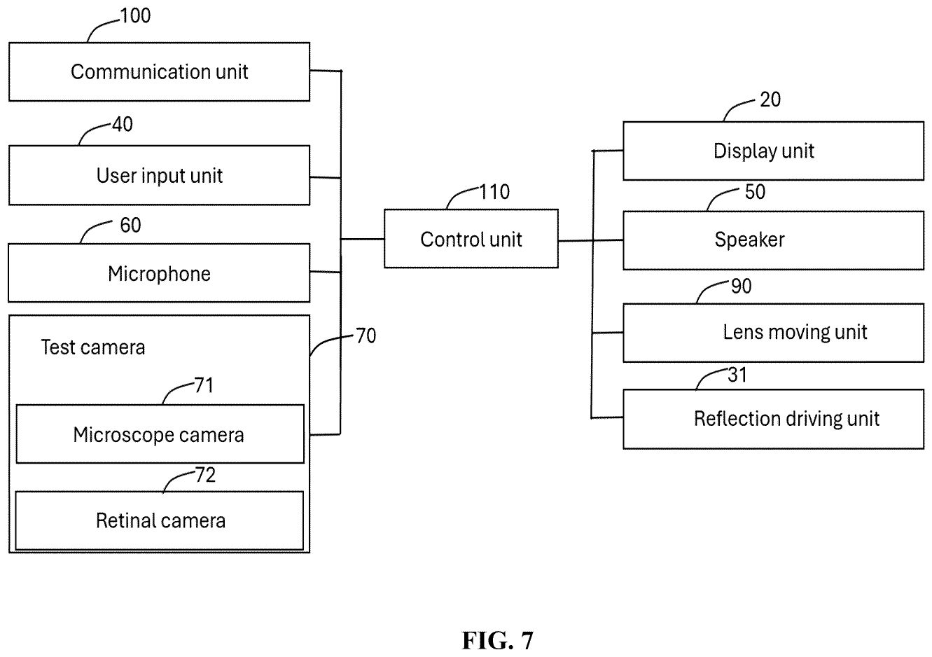

FIG. 1 is a view illustrating a user wearing a portable optometry device 1 according to the present invention, FIG. 2 is a view illustrating a screen light of an eyesight test image from a display unit 20 reaching the user's eye, FIG. 3 is a view illustrating an example of providing eyesight test guidance via voice, FIG. 4 is a view illustrating a test camera 70 for capturing the user's eye, FIG. 5 is a view illustrating the operation that a lens 82 moves, FIG. 6 is a view illustrating an eyesight test conducted using voice, and FIG. 7 is a control block diagram of the portable optometry device 1.

The configuration of the portable optometry device 1 will be described with reference to FIGS. 1 to 7.

The portable optometry device 1 includes an optometry case 10, a display unit 20, a reflection unit 30, a user input unit 40, a speaker 50, a microphone 60, a test camera 70, a lens unit 80, a lens moving unit 90, a communication unit 100, and a control unit 110.

The optometry case 10 includes a case body 11, a face support part 12, a transparent window 13, and a fastening band 14.

The case body 11 has a thick goggle-like shape worn on a user's face and includes an internal accommodation space.

The face support part 12 includes an internal accommodation space and is seated to cover the user's eyes.

The transparent window 13 is supported by the case body 11 and may be arranged in an area of the face support part 12.

The fastening band 14 may be formed of a band coupled to one side and the other side of the case body 11 and configured to be supported on the back of the user's head. The fastening band 14 may have elasticity, such as a rubber band.

The display unit 20 is arranged on one side of the internal accommodation space of the optometry case 10 and provides a screen for an eyesight test. The display unit 20 displays images based on video signals processed by image processing. The implementation method of the display unit 20 is not limited, and may be implemented using various display types, such as a liquid crystal display, plasma display, light-emitting diode (LED), organic light-emitting diode (OLED), surface-conduction electron-emitter, carbon nanotube, and nano-crystal display.

The display unit 20 may additionally include further components depending on the implementation method. For example, in the case where the display unit 20 is a liquid crystal type, the display unit 20 may include a liquid crystal display panel (not shown), a backlight unit (not shown) supplying light thereto, and a panel driving board (not shown) for driving the panel (not shown). The display unit 20 may display voice recognition results as information regarding the recognized voice. Here, the voice recognition results may be displayed in various forms such as text, graphics, or icons, and the text may include letters and numbers. The display unit 20 may further display candidate commands and application information based on the voice recognition result.

The user can check whether the voice is properly recognized based on the voice recognition result displayed on the display unit 20, and may select a command corresponding to the spoken voice among the displayed candidate commands by operating the user input unit 40 provided on a remote controller, or may select and confirm information related to the voice recognition result.

The reflection unit 30 reflects the screen light output from the screen of the display unit 20 toward the user's eye. The reflection unit 30 may be provided in plurality and arranged at a plurality of positions within the internal accommodation space of the optometry case 10. The reflection unit 30 includes a reflection driving unit 31.

The reflection driving unit 31 is configured to rotate and move the reflection unit 30 with respect to the case body 11.

The user input unit 40 is configured to receive a user command. The user input unit 40 may receive a user's touch input or remote input via a remote controller and transmit the user input to the control unit 110. Additionally, the user input unit 40 may also receive a voice input from the user and transmit the corresponding voice signal to the control unit 110. In this case, the user input unit 40 may be implemented, for example, as a microphone. The user input unit 40 may also perform signal processing on the received voice signal. However, the types of user inputs receivable by the user input unit 40 are not limited thereto, and other types of user inputs such as motion recognition may also be accepted.

The speaker 50 is configured to output eyesight test guidance as voice.

The microphone 60 is configured to collect the user's voice.

The test camera 70 is provided within the internal accommodation space of the optometry case 10 and is configured to capture an image of the user's eye. The test camera 70 includes a microscope camera 71 and a retinal camera 72.

The microscope camera 71 may be a camera equipped with a slit-lamp microscope for inspecting the surface of the eye.

The retinal camera 72 may be a camera that enables examination of the retina and optic nerve of the eye to diagnose conditions such as arteriosclerosis, diabetic retinopathy, and hypertension.

The lens unit 80 includes a convex lens 81 and a concave lens 82.

The convex lens 81 is arranged on an output path of the screen light from the display unit 20 to enlarge the eyesight test image. The convex lens 81 may be movable relative to the optometry case 10.

The concave lens 82 is arranged on the output path of the screen light from the display unit 20 to reduce the eyesight test image. The concave lens 82 may also be movable relative to the optometry case 10.

The lens moving unit 90 is configured to move the convex lens 81 and the concave lens 82.

The communication unit 100 is capable of performing wireless communication. The wireless communication may include at least one of infrared communication, RF, Zigbee, and Bluetooth. The communication unit 100 receives video signals and transmits the video signals to the control unit 110 described below. The communication unit 100 may be implemented in various forms depending on the format of the received video signal and the configuration of the user terminal. For example, the communication unit 100 may wirelessly receive a radio frequency (RF) signal transmitted from a broadcasting station (not shown), or receive a video signal via a wired connection according to composite video, component video, super video, SCART, or HDMI (High-Definition Multimedia Interface) standards. When the video signal is a broadcast signal, the communication unit 100 may include a tuner for tuning broadcast channels.

The control unit 110 stores eyesight test images including guidance for conducting the user's eyesight test and controls the display unit 20 to output the eyesight test image when a user command for the eyesight test is input through the user input unit 40.

The control unit 110 may control the speaker 50 to output the eyesight test guidance as voice corresponding to the portion of the eyesight test image displaying the eyesight test guidance.

The control unit 110 may control the display unit 20 such that a predetermined screen light output intensity of the display unit 20 is provided based on the distance of the plurality of reflection units 30.

The control unit 110 stores a plurality of disease reference images corresponding to different ocular diseases and compares the user's eye image captured by the test camera 70 with the plurality of stored disease images. Based on the comparison, the control unit 110 may identify a disease image having a similarity above a predetermined threshold.

Here, the control unit 110 may transmit the image of the user's eye captured by the test camera 70 to a predetermined terminal of an ophthalmologist so that a diagnosis can be performed.

The control unit 110 may control the lens movement driving unit 90 to adjust the object size of the eyesight test image for the user's eyesight test.

The control unit 110 stores at least one voice data corresponding to each of the plurality of objects in the eyesight test image, controls the output of the eyesight test image, compares the voice data corresponding to the output object with the user's voice collected by the microphone 60, and controls the device to display a next object smaller than the previously displayed object is output if the comparison result indicates a match.

Here, the user can download and install an application for an eyesight test on a user terminal, such as a smartphone, and pairing is achieved between the user terminal and the communication unit 100, so that the eyesight test image and the eyesight test guidance are output to perform the eyesight test. In this case, the voice output and collection may be handled by the user terminal. If no user terminal is available, the eyesight test may be performed independently. Even when the eyesight test is conducted independently, the test information may be set to be transmitted to the user terminal and a predetermined terminal of a physician or a guardian.

FIG. 2 is a view illustrating a case where the screen light of the eyesight test image from the display unit 20 reaches the user's eye.

The screen light of the eyesight test image output from the display unit 20 is reflected by the reflection unit 30 below the display unit 20 and the reflection unit 30 on the left of the display unit 20, and then reaches the user's eye through the transparent window 13. As described above, a sufficient testing distance can be secured through two reflections.

FIG. 3 is a view illustrating an example of providing eyesight test guidance via voice.

When the user wears the portable optometry device 1 and starts the eyesight test, the eyesight test image shows a phrase such as “Please close your left eye” and the same content is also output as voice guidance to proceed with the test.

FIG. 4 is a view illustrating the test camera 70 that captures the user's eye.

The screen light of the eyesight test image output from the display unit 20 is reflected by the reflection unit 30 below the display unit 20 and the reflection unit 30 on the left of the display unit 20, and then reaches the user's eye through the transparent window 13. On the left side, the microscope camera 71 and the retinal camera 72 are arranged to capture the user's eye. Eye imaging may be performed during the eyesight test, or may be immediately performed for the purpose of diagnosing eye diseases. When eye imaging is performed, the display unit 20 may output a bright screen light to facilitate proper imaging of the eye.

FIG. 5 is a view illustrating the operation of moving the lenses 81 and 82.

In FIG. 5, the concave lens 82 is moved from position “a” to position “b”. Accordingly, the size of the object in the eyesight test image is reduced, and such adjustment is used to perform the eyesight test.

FIG. 6 is a view illustrating an eyesight test conducted using voice.

During the eyesight test, when the user reads an object (A) in the test image, the system analyzes the voice and determines whether the voice matches the displayed object. If a match is found, the user's visual acuity at the current level is considered verified, and the system displays the next smaller object to continue the test.

In addition to the foregoing embodiment, alternative embodiments of the present invention will now be described.

The display unit may be arranged on the inner upper side of the internal accommodation space of the case body while the user is wearing the optometry case, and the reflection units may be disposed on the lower side and on the side farther from the user's eye such that the screen light of the display unit is reflected by the lower reflection unit and then reflected by the reflection unit located farther from the user's eye, thereby directing the light toward the user's eye.

Here, the display unit may be arranged on either the inner right side or inner left side of the internal accommodation space of the case body while the user is wearing the optometry case, and the reflection units may be arranged on the opposite side, the inner upper side, the lower side, and the side farther from the user's eye such that the screen light of the display unit is first reflected by the reflection unit disposed on either the inner right side or inner left side, and then reflected by another reflection unit arranged on the opposite side, the inner upper side, the lower side, or the side farther from the user's eye, thereby being directed toward the user's eye. In this manner, a longer eyesight testing distance can be secured.

The lens may be arranged between the display unit and the reflection unit or between reflection units to secure a sufficient distance for the screen light to reach the user's eye. One lens may be arranged, or two lenses may be arranged depending on the size of the internal space of the case body.

The control unit may rotate the reflection unit located farther from the user's eye so that the screen light of the eyesight test image is directed to the user's left eye when testing the left eye, and likewise directed to the right eye when testing the right eye by rotating the reflection unit accordingly.

The test camera may also be rotatably coupled to the case body to allow for the test of both the left and right eyes of the user, and may further include a camera rotation unit for rotating the test camera with respect to the case body.

The control unit may store contact lens information corresponding to different levels of eyesight and provide the appropriate contact lens information to the user upon completion of the eyesight test.

The control unit may train an artificial intelligence-based disease estimation model through machine learning to output disease diagnosis by image. The AI-based disease estimation model may be an algorithm that receives first and second fundus images to output a value corresponding to the type of disease. The algorithm may be one of an artificial neural network (ANN), support vector machine (SVM), decision tree, and random forest. The artificial neural network is mainly used in deep learning and is a statistical learning algorithm inspired by machine learning and biological neural networks, and may be a convolutional neural network including a feature extraction network and a classification network. In this instance, the convolutional neural network, which is a type of deep, feedforward ANN used for analyzing visual images, may be divided into a feature extraction stage and a classification stage. The convolutional neural network is capable of extracting features from a specific image and recognizing the image based on the extracted features.

According to the present invention, the portable optometry device 1 can secure a viewing distance by reflecting an eyesight test image output from a display unit by a reflection unit to reduce the size and length of the optometry device compared to conventional optometry instruments, thereby enhancing user portability.

In addition, the portable optometry device can provide an eyesight test guidance via voice output, thereby improving user convenience during the test.

Furthermore, the portable optometry device includes the plurality of reflection units, thereby increasing the intensity of screen light output in proportion to the distance the screen light of the eyesight test image travels to reach the user's eyes.

Moreover, the portable optometry device can capture images of the user's eye during the eyesight test, and identify ocular diseases using the captured eye image.

Additionally, Although it may be difficult to secure a sufficient distance for the screen light of the eyesight test image to reach the user's eyes due to a small internal accommodation space, such distance can be effectively secured by using a lens unit.

In addition, the portable optometry device can show the user objects while enlarging and reducing object sizes in the eyesight test image by moving the lens, thereby allowing for an accurate eyesight test.

Furthermore, since the user reads the objects displayed in the eyesight test image, the eyesight test can be conducted smoothly and conveniently.

What is claimed is:

1. A portable optometry device, the device comprising:
an optometry case having an internal receiving space and a face mounting portion that covers and rests on a user's eyes;
a display unit disposed on one side of the internal receiving space of the optometry case to provide a screen for vision test;
a reflection unit that reflects screen light output from the screen of the display unit to the user's eyeball;
a user input unit that inputs a user command; and
a control unit that stores a vision test image including a vision test guide for the user's vision test, and controls, when a user command for vision test is input to the user input unit, the display unit to output the vision test image.

2. The device of claim 1, further comprising:
a speaker that outputs the vision test guide in a voice,
wherein the control unit controls the speaker to output the vision test guide in a voice in response to the vision test guide being displayed in the vision test image.

3. The device of claim 1, wherein the reflection unit is provided in plurality in a plurality of positions in the internal receiving space of the optometry case, and
wherein the control unit controls the display unit to allow preset screen light output intensities of the display unit corresponding to distances of the plurality of reflection units to be output.

4. The device of claim 1, further comprising:
a test camera disposed in the internal receiving space of the optometry case to capture the user's eyeball,
wherein the control unit stores a plurality of disease images for a plurality of eyeball diseases, compares the user's eyeball image captured by the test camera with the plurality of disease images, and identifies disease images above a preset similarity level as a result of the comparison.

5. The device of claim 1, further comprising:
a lens unit including a convex lens and a concave lens disposed on an output path of screen light of the display unit to enlarge and reduce the vision test image.

6. The device of claim 1, further comprising:
a microphone that collects the user's voice,
wherein the control unit stores at least one voice for each of a plurality of objects in the vision test image, controls the vision test image to be output, then compares the voice for the output object with the user voice collected by the microphone, and controls, when it is determined to be the same as a result of the comparison, a next object smaller than the output object to be output.
